(19) United States
(12) Patent Application Publication
(10) Pub. No.: US 2021/0230656 A1
(43) Pub. Date: Jul. 29, 2021

(54) PRODUCTION OF OLIGOSACCHARIDES

(71) Applicant: DSM IP Assets B.V., TE Heerlen (NL)

(72) Inventors: Arthur Maurits Christiaan JANSE, Echt (NL); Michael Benjamin JOHNSON, Columbia, MD (US)

(21) Appl. No.: 17/057,393

(22) PCT Filed: May 15, 2019
(86) PCT No.: PCT/US2019/032396

§ 371 Date: Nov. 20, 2020

Related U.S. Application Data
(60) Provisional application No. 62/675,393, filed on May 23, 2018.

(51) Int. Cl. C12P 19/02; C12P 19/14; A23L 33/21; A23L 33/00; A23K 10/14; A23K 10/16; A23K 20/163; A23L 5/40; B01D 15/18; B01D 15/36; B01D 61/14; B01D 61/02; B01D 61/58

(52) U.S. Cl. CPC C12P 19/02; B01D 2311/2623; A23L 33/21; A23L 33/40; A23K 10/14; A23K 10/16; A23K 20/163; A23L 5/40; B01D 15/1821; B01D 15/362; B01D 61/145; B01D 61/027; B01D 61/58; A23V 2002/00; B01D 2311/2688; B01D 2311/2676; B01D 2311/2649; C12P 19/14

(57) ABSTRACT

A method for producing and purifying human milk oligosaccharides (HMOs) is provided. The method includes fermentation of a genetically modified microbial organism, preferably a genetically modified yeast strain, and downstream processing of the fermentation product using one or more of an enzymatic treatment, filtration, and a simulated moving bed (SMB) chromatography step. Use of the resulting HMO in food or feed applications, preferably in infant food and/or formula is also provided.

### CROSS-REFERENCE TO RELATED APPLICATIONS

This application claims the benefit of the filing date of U.S. Provisional Patent Application No. 62/675,393 filed May 23, 2018, the disclosure of which is hereby incorporated by reference in its entirety.

### BACKGROUND

The present disclosure relates to processes for producing and purifying human milk oligosaccharides (HMOs). The process includes fermentation of a genetically modified microbial organism, preferably a genetically modified yeast strain, and downstream processing of the fermentation product using one or more of an enzymatic treatment, filtration and single column chromatography or multicolumn chromatography, in particular, in simulated moving bed (SMB) chromatography mode.

Human milk contains a family of unique oligosaccharides, HMOs, which are structurally diverse unconjugated glycans. Despite being the third most abundant solid component of human milk (after lactose and fat), human infants cannot actually digest HMOs. Instead, they function as prebiotics to help establish commensal bacteria. HMOs also function as anti-adhesives that help prevent the attachment of microbial pathogens to mucosal surfaces. The occurrence and concentration of these complex oligosaccharides are specific to human and are not found in large quantities in the milk of other mammals such as domesticated dairy animals.

Due to the challenges involved in the chemical synthesis of human milk oligosaccharides, several enzymatic methods and fermentative approaches have been developed, primarily in bacterial strains such as E. coli. However, these methods yield complex mixtures of oligosaccharides, i.e., the desired product is contaminated with starting material such as lactose, biosynthetic intermediates, and substrates such as individual monosaccharides, polypeptides, etc.

Processes in the state of the art for purifying individual oligosaccharide products from these mixtures are technically complex. Multiple crystallization operations are used in the sugar industry to separate disaccharides such as lactose or sucrose from complex mixtures such as whey or molasses. The disadvantage of these methods is that they are elaborate and produce low yield. For the purification of complex oligosaccharides, such as certain HMOs, size exclusion chromatography has been most widely used. However, size exclusion chromatography is not economical for food applications and certain HMOs, e.g., 2′-fucosyllactose (2′FL), cannot be produced in adequate amounts.

Thus, there remains a need to provide improved processes for the production and purification of HMOs, and in particular 2′FL, at lower costs and higher efficiency, and/or purity.

The solution to this technical problem is provided by the embodiments characterized below.

### BRIEF SUMMARY

The present application provides methods for producing and purifying human milk oligosaccharides (HMOs). In particular, the method of the invention includes fermentation of a microbial organism that has been genetically modified to produce the desired HMO in a suitable fermentation medium and purification of the resulting fermentation product to remove by-products and obtain the desired HMO.

In some embodiments, the microbial organism is a yeast that has been genetically modified to produce the desired HMO.

The desired HMO, such as 2′-fucosyllactose (2′FL), is purified from the fermentation medium by simulated moving bed (SMB) chromatography. After fermentation, a fermentation medium containing the desired HMO is applied to the SMB chromatography. Preferably, before applying the fermentation medium to the SMB chromatography, the fermentation medium is subjected to one or more of the following:

i) enzymatic treatment of the fermentation product;

ii) removal of the biomass;

iii) ultrafiltration of the fermentation product;

and/or iv) nanofiltration of the fermentation product.

In some embodiments, the enzymatic treatment of the fermentation product is used to convert lactose and/or sucrose to monosaccharides

In some embodiments, the enzymatic treatment comprises incubation of the fermentation product with one or more enzymes. In some embodiments, the enzyme is a lactase, a β-galactosidase, a trehalase, and/or an invertase.

In some embodiments, removal of the biomass is performed by centrifugation, filtration, ultrafiltration, nanofiltration, or combinations thereof.

In some embodiments, removal of the biomass is performed by centrifugation.

In some embodiments, removal of the biomass is performed by filtration.

In some embodiments, ultrafiltration of the fermentation product is used to remove proteins and/or other high molecular weight molecules such as DNA.

In some embodiments, nanofiltration of the fermentation product is used to remove low molecular weight molecules, such as oligosaccharides larger than the target compound and/or smaller sugar components and peptides.

In some embodiment, the fermentation product is subjected to more than one nanofiltration step. For example, a first nanofiltration step may be performed to remove molecules that are slightly larger or larger than the desired HMO, such as, for example, larger oligosaccharides. A second nanofiltration step may be performed to remove molecules that are smaller than the desired HMO, such as, for example, mono-saccharides, amino acids, and ions. In some embodiments, the nanofiltration steps are performed consecutively.

In some embodiments, the method of the invention further comprises one or more of the following:
a) decolorization;
b) additional filtration; and/or
c) concentrating and/or drying the resulting HMO solution.

In a preferred embodiment, decolorization, additional filtration, and/or drying is performed after the SMB chromatography step.

It will be understood that the steps of the method of the invention, with the exception of the drying step, may be performed in any order. In some embodiments, one or more steps of method of the invention may be performed more than once. In a preferred embodiment, the steps of the method of the invention are performed in the order listed above.

Also provided is the HMO obtained according to the method of the invention.

In some embodiments, the HMO obtained according to the method of the invention is in a food, supplement, or pharmaceutical composition. The pharmaceutical composition can contain a pharmaceutically acceptable carrier.

In some embodiments, the HMO obtained according to the method of the invention can be used in a food product. A food product is any food for non-human animal or human consumption, and includes both solid and liquid compositions. A food product can be an additive to animal or human foods. Foods include, but are not limited to, common foods; liquid products, including milks, beverages, therapeutic drinks, and nutritional drinks; functional foods; supplements; nutraceuticals; infant formulas, including formulas for pre-mature infants; foods for pregnant or nursing women; foods for adults; geriatric foods; and animal foods.

### BRIEF DESCRIPTION OF THE DRAWINGS

For a further understanding of the nature, objects, and advantages of the present disclosure, reference should be made to the following detailed description, read in conjunction with the following drawings, wherein like reference numerals denote like elements.

FIG. 1 shows exemplary steps of the invention.

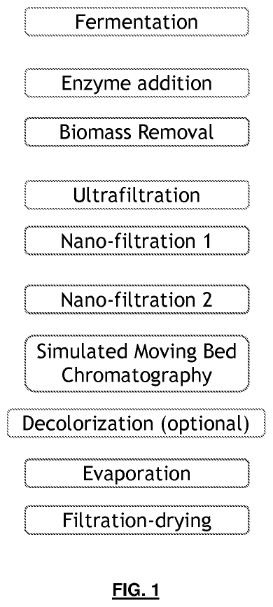

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

The term “fermentation product”, as used herein, refers to the product obtained from fermentation of the microbial organism. Thus, the fermentation product comprises cells, the fermentation medium, residual substrate material, and any molecules/by-products produced during fermentation, such as the desired HMO. After each step of the purification method, one or more of the components of the fermentation product is removed, resulting in a more purified HMO.

The subject disclosure features, in one aspect, a method for producing and purifying human milk oligosaccharides comprising one or more of the following: fermentation of a genetically modified microbial organism; enzymatic treatment to convert lactose and sucrose to monosaccharides; centrifugation and/or filtration to remove biomass (e.g., cells, high molecular weight molecules); ultrafiltration to remove proteins and/or other higher molecular weight molecules such as DNA; one or more nanofiltration steps to remove molecules that are slightly larger and/or smaller than the desired HMO; and simulated moving bed (SMB) chromatography.

The desired HMO produced and purified according to the method of the invention is selected from the group consisting of: 2′-fucosyllactose, 3-fucosyllactose, 2′,3-difucosyllactose, lacto-N-triose II, lacto-N-tetraose, lacto-N-neotetraose, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In a preferred embodiment, the desired HMO produced and purified according to the method of the invention is 2′-fucosyllactose (2′FL).

The desired HMO, such as 2′FL, is produced by fermentation of a genetically modified microbial organism. Fermentation may be performed in any suitable fermentation medium, such as, for example, a chemically defined fermentation medium. The fermentation medium may vary based on the microbial organism used.

In a preferred embodiment, the microbial organism is a genetically modified yeast. The yeast may be, for example, a Saccharomyces strain, a Candida strain, a Hansenula strain, a Kluyveromyces strain, a Pichia strain, a Schizosaccharomyces stain, a Schwanniomyces strain, a Torulaspora strain, a Yarrowia strain, or a Zygosaccharomyces strain.

In some embodiments, the yeast is, for example, Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, or Zygosaccharomyces bailii.

Separation of biomass from the fermentation product can be performed by any suitable means. In one embodiment, the fermentation product is centrifuged to separate and remove the biomass. In another embodiment, the fermentation product is filtered to separate and remove the biomass. In some embodiments, a combination of centrifugation and filtration is used to separate and remove the biomass from the fermentation product. In some embodiments, the fermentation medium is filtered using membrane filtration. In a preferred embodiment, the membrane filtration is microfiltration, ultrafiltration, or combinations thereof.

In a preferred embodiment, the fermentation product is filtered through a cross flow microfiltration, preferably with a cut off of 10 microns, preferably with a cut off of 5 microns, preferably with a cut off of 0.2 microns, to separate and remove the biomass.

In some embodiments, ultrafiltration is performed to remove proteins and other high molecular weight compounds, such as DNA, from the fermentation product. In some embodiments, the pore size of the ultrafiltration membrane is 100 kD molecular weight cut-off (“MWCO”) or less, 90 kD MWCO, 80 kD MWCO, 70 kD MWCO, 60 kD MWCO, 50 kD MWCO, 40 kD MWCO, 35 kD MWCO, 30 kD MWCO, 25 kD MWCO, 20 kD MWCO, 15 kD MWCO, 10 kD MWCO, 9 kD MWCO, 8 kD MWCO, 7 kD MWCO, 6 kD MWCO, or 5 kD MWCO or less.

In some embodiments, a first nanofiltration step is performed to remove molecules having a slightly larger molecular weight than the desired HMO from the fermentation product. In some embodiments, the pore size of the nanofiltration membrane is 0.5 kD MWCO, 0.6 kD MWCO, 0.7 kD MWCO, 0.8 kD MWCO, 0.9 kD MWCO, 1 kD MWCO, 1.2 kD MWCO, 1.4 kD MWCO, 1.6 kD MWCO, 1.8 kD MWCO, 2 kD MWCO or more, or has a MDWCO higher than the target molecule of interest.

In some embodiments, a second nanofiltration step is performed to remove low molecular weight molecules such as mono-saccharides and ions. In some embodiments, the pore size of the second nanofiltration membrane is 500 dalton (Da) or less molecular weight cut-off (“MWCO”), 450 Da MWCO, 400 Da MWCO, 350 Da MWCO, 300 Da MWCO, 250 Da MWCO, or 200 Da MWCO or less.

Using the ultrafiltration and nanofiltration techniques described herein, two separate streams are produced after each filtration, one stream known as a retentate and a second stream known as a permeate.

In some embodiments, the yield of the desired HMO in the permeate after an ultrafiltration step is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

In some embodiments, the yield of the desired HMO in the permeate after a nanofiltration step is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

In some embodiments, the yield of the desired HMO in the retentate after a nanofiltration step is greater than 50%, greater than 60%, greater than 70%, greater than 80%, greater than 90%, greater than 91%, greater than 92%, greater than 93%, greater than 94%, greater than 95%, greater than 96%, greater than 97%, greater than 98%, or greater than 99%.

The yield (Y) in the retentate is calculated by: Yᵣ=CᵣVᵣ/C_{f}V_{f} where Cᵣ is the concentration of that solute in the retentate, C_{f} is the concentration of that solute in the initial feed, Vᵣ is the volume of the retentate, and V_{f} is the volume of the initial feed.

The yield (Y) in the permeate is calculated by: Yₚ=CₚVₚ/C_{f}V_{f} where Cₚ is the concentration of that solute in the permeate, C_{f} is the concentration of that solute in the initial feed, Vₚ is the volume of the permeate, and V_{f} is the volume of the initial feed.

In some embodiments, a desalting step is performed. This step may use a membrane and/or an electrodialysis step. The desalting step may also be the same as the second nanofiltration step.

The process of the invention includes subjecting the fermentation product to simulated moving bed (SMB) chromatography to purify the desired HMO, such as 2′FL, from impurities, other similar molecules, undesired molecules, and other charged molecules. SMB chromatography is preferably performed after one or more filtration steps. In a preferred embodiment, the fermentation product is subjected to centrifugation, microfiltration, ultrafiltration, and one or more nanofiltration steps prior to performing SMB chromatography.

The SMB chromatography step may comprise:

i) at least 4 columns, preferably at least 8 columns, more preferably at least 12 columns, wherein at least one column comprises a weak or strong cation exchange resin, preferably a cation exchange resin in the H⁺-form or Ca²⁺-form; and/or

ii) four zones I, II, III and IV with different flow rates; and/or

iii) an eluent comprising or consisting of water, preferably ethanol and water, more preferably 5-15 vol.-% ethanol and 85-95 vol.-% water, most preferably 9-11 vol.-% ethanol and 89-91 vol.-% water, wherein the eluent optionally further comprising sulfuric acid, preferably 0 mM sulfuric acid; more preferably 2-5 mM sulfuric acid; and/or

iv) an operating temperature of 15° to 60° C., preferably 20° to 55° C., more preferably 25° to 50° C.

If the HMO to be purified is 2′FL, the SMB chromatography step may comprise

i) four zones I, II, III and IV with different flow rates, wherein the flow rates are preferably: 28-32 ml/min in zone I, 19-23 ml/min in zone II, 21-25 ml/min in zone III and/or 16-20 ml/min in zone IV; and/or

ii) a feed rate of 2-4 ml/min, preferably 3 ml/min; and/or

iii) an eluent flow rate of 10-13 ml/min, preferably 11.5 ml/min; and/or

iv) a switching time of 16-20 min, preferably 17-19 min, more preferably 18 min.

Preferably, at least one of the columns comprises 0.1 to 5000 kg of cation exchange resin, preferably 0.2 to 500 kg of cationic exchange resin, more preferably 0.5 to 50 kg of cation exchange resin, most preferably 1.0 to 20 kg of cation exchange resin.

The amount of cation exchange material, the flow rate in the different zones, the feed rate, the eluent flow rate, and/or the switching time may be scaled up as needed. The scaling-up may be by a factor of 2, 3, 4, 5, 6, 7, 8, 9, 10, 20, 30, 40, 50, 60, 70, 80, 90, 100, 1000 or all possible scaling factors in between said values.

In the columns, a strong cation exchange resin may be used as stationary phase. Preferably, the cation exchange resin is a sulfonic acid resin, more preferably a Purolite® PCR833H (Purolite, Ratingen, Germany), Lewatit MDS 2368 and/or Lewatit MDS 1368 resin. If a cation ion exchange resin is employed in the columns, it may be regenerated with sulfuric acid. Sulfuric acid can be employed in the eluent, preferably at a concentration of 10 mM sulfuric acid or less. The (strong) cation exchange resin may be present in H⁺-form or in Ca²⁺-form.

In some embodiments, the percent purity of the HMOs produced is greater than or equal to 88%. In some embodiments, the purity of the HMOs produced is greater than or equal to 90%, greater than 91%, greater than 92%, greater than 93%, greater than 94%, greater than 95%, greater than 96%, greater than 97%, greater than 98%, greater than 99%. The following formula is used:

Percent purity=(C_{HMO}/C_{DM})*100 wherein CHMO is the concentration of the desired HMO and CDM is the concentration of total dry matter.

In some embodiments, the process of the invention includes one or more decolorization steps. Decolorization can be performed by any suitable means. For example, decolorization can be performed by treatment of the fermentation product with activated carbon. The one or more decolorization steps may be performed at any point during the process of the invention. In a preferred embodiment, decolorization is performed after SMB chromatography.

The resulting solution containing the desired HMO may be concentrated and/or dried. In some embodiments, the resulting solution is evaporated, freeze dried, or any combination thereof.

The HMOs obtained using the process of the invention are suitable for use in food and feed applications. In some embodiments, the obtained HMOs are used in infant food, infant formula, and/or infant supplements. In a preferred embodiment, the obtained HMOs are used in human infant food, human infant formula, and/or human infant supplements.

In other embodiments, the HMOs obtained using the process of the invention are used in medicine, such as, for example, as a treatment for a gastrointestinal disorder and/or as a prebiotic.

The following examples are offered to illustrate, but not to limit, the claimed invention.

### EXAMPLES

### Example 1

Microbial cells that have been genetically modified to express one or more human milk oligosaccharides are cultured in a suitable fermentation medium, resulting in a fermentation product containing the human milk oligosaccharides. This fermentation product is then treated with one or more enzymes, such as lactase, β-galactosidase, trehalase, and/or invertase. Biomass, such as cells, is removed from the fermentation product using any suitable means, such as centrifugation followed by filtration, or any combinations thereof. The fermentation product is then subjected to ultrafiltration to remove proteins and other high molecular weight compounds, such as DNA. A nanofiltration step is then performed to remove molecules having a slightly larger molecular weight than the desired HMO(s) from the fermentation product. A second nanofiltration step is performed to remove low molecular weight molecules, such as mono-saccharides and ions.

Next, the fermentation product is subjected to a simulated moving bed (SMB) chromatography step to further purify the desired HMO(s). The system used for the SMB chromatography contains at least 4 columns containing a cation exchange resin. The flow rates used in the different zones are 28-32 ml/min in zone I, 19-23 ml/min in zone II, 21-25 ml/min in zone III and/or 16-20 ml/min in zone IV. The eluent used can be 10% ethanol in water. The resulting solution containing the purified HMO(s) can optionally be subjected to decolorization, filtration, and/or drying.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A method for the production of one or more human milk oligosaccharides (HMOs) comprising:
a) fermentation of a microbial organism that has been genetically modified to produce one or more HMOs in a suitable fermentation medium to form a fermentation product;
b) enzymatic treatment of the fermentation product;
c) removal of the biomass from the fermentation product;
d) ultrafiltration;
e) nanofiltration; and
f) a column chromatography step.

2. The method of claim 1, wherein the one or more HMOs is 2′-fucosyllactose.

3. The method of claim 1 or 2, wherein the microbial organism is a yeast.

4. The method of claim 3, wherein the yeast is selected from the group consisting of: Saccharomyces, Candida, Hansenula, Kluyveromyces, Pichia, Schizosaccharomyces, Schwanniomyces, Torulaspora, Yarrowia, and Zygosaccharomyces.

5. The method of claim 3 or 4, wherein the yeast is selected from the group consisting of: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii.

6. The method of any of claims 1-5, wherein the enzymatic treatment comprises incubation of the fermentation product with one or more enzymes selected from the group consisting of: lactase, β-galactosidase, trehalase, and invertase.

7. The method of any of claims 1-6, wherein the enzymatic treatment converts lactose and/or sucrose to monosaccharides.

8. The method of any of claims 1-7, wherein the removal of the biomass from the fermentation product comprises centrifugation, filtration, or combinations thereof.

9. The method of any of claims 1-8, wherein the column chromatography step is a single column or a multiple column.

10. The method of any of claims 1-9, wherein the column chromatography step is simulated moving bed chromatography.

11. The method of any of claims 1-10, wherein the nanofiltration step is performed more than once.

12. The method of any of claims 1-11, wherein the nanofiltration is performed twice.

13. The method of claim 12, wherein the nanofiltration steps are performed consecutively.

14. The method of any of claims 1-13, wherein the method further comprises one or more of:
a) decolorization;
b) filtration; and/or
c) drying.

15. The method of claim 14 wherein the drying comprises evaporation.

16. The method of any of claims 10-15, wherein the simulated moving bed chromatography comprises
i) at least 4 columns, wherein at least one column comprises a weak or strong cation exchange resin; and/or
ii) four zones I, II, III and IV with different flow rates; and/or
iii) an eluent comprising water; and/or
iv) an operating temperature of 15° to 60° C.

17. The method of claim 16, wherein the eluent further comprises ethanol and/or sulphuric acid.

18. The method of claim 16 or 17, wherein the flow rate in zone I is 28-32 ml/min, the flow rate in zone II is 19-23 ml/min, the flow rate in zone III is 21-25 ml/min, and/or the flow rate in zone IV is 16-20 ml/min.

19. The method of any of claims 16-18, wherein the operating temperature is from 25° to 50° C.

20. The method of any of claims 16-19, comprising a feed rate of 2-4 ml/min.

21. The method of any of claims 16-20 comprising an eluent flow rate of 10-13 ml/min.

22. The method of any of claims 16-21, comprising a switching time of 16-20 minutes.

23. The method of any of claims 16-22, wherein at least one column comprises 0.1 to 5000 kg of cation exchange resin.

24. The method of any of claims 16-23, wherein the cation exchange resin is a sulfonic acid resin.

25. The method of any of claims 1-24 wherein a)-f) are performed in any order.

26. The method of any of claims 1-25, wherein a)-f) are performed in the order provided in claim 1.

27. The HMO obtained according to the method of any of claims 1-26.

28. Use of the HMO obtained according to the method of any of claims 1-26 in a food or feed preparation.

29. The use according to claim 28, wherein the food is a human food.

30. The use according to claim 29, wherein the food is an infant food.

31. The use according to claim 29 or 30, wherein the food is an infant formula or an infant supplement.

32. Use of the HMO obtained according to the method of any of claims 1-26 in a dietary supplement.
